(19) United States
(12) Patent Application Publication
(10) Pub. No.: US 2021/0120855 A1
(43) Pub. Date: Apr. 29, 2021

(54) MIXED SACCHARIDE COMPOSITION COMPRISING MALTOOLIGOSACCHARIDE

(71) Applicant: SAMYANG CORPORATION, Seoul (KR)

(72) Inventors: Ji Won PARK, Seongnam-si (KR); Sung Won PARK, Seoul (KR); Eunsoo CHOI, Seongnam-si (KR); Go-Eun KIM, Gwangju-si (KR); Kyung-Hun RYU, Seongnam-si (KR); Chong Jin PARK, Seoul (KR); Jae-Kyung YANG, Hwaseong-si (KR); Tae-Chul HAN, Yongin-si (KR); Bong Chan KIM, Seoul (KR); Yong-In KIM, Yongin-si (KR); II SEO, Gwangju-si (KR); Seung Mi LEE, Hwaseong-si (KR); Hye Jin LIM, Suwon-si (KR)

(21) Appl. No.: 17/254,410

(22) PCT Filed: Jun. 28, 2019
(86) PCT No.: PCT/KR2019/007911

§ 371 Date: Dec. 21, 2020

(30) Foreign Application Priority Data
Jun. 28, 2018 (KR) 10-2018-0075049
Nov. 1, 2018 (KR) 10-2018-0133114

(51) Int. Cl. A23L 27/30; C07H 3/06; A23L 29/30; A23L 3/46; A23G 9/34; A23C 13/12; A23L 2/60

(52) U.S. Cl. CPC A23L 27/33; C07H 3/06; A23L 29/30; A23V 2002/00; A23G 9/34; A23C 13/12; A23L 2/60; A23L 3/46

(57) ABSTRACT

The present disclosure relates to a mixed saccharide including maltooligosaccharide, which has less saccharide content and calories than conventional maltooligosaccharide, glucose syrup, maltose syrup and low DE glucose syrup, and has reached an equivalent level of viscosity as a conventional starch syrup.

### TECHNICAL FIELD

The present disclosure relates to a mixed saccharide composition containing maltooligosaccharide and a food composition containing the same. The present disclosure has an advantage that the content of saccharide such as glucose, fructose, maltose or sucrose in the mixed saccharide composition is as low as 10% or less relative to the solid content, and calories can also be reduced by 15-30%, and also has an advantage that the food texture and/or physical properties when applied to foods can be improved.

### BACKGROUND ART

Recently, processed foods with high saccharide content have been frequently criticized due to their association with various adult diseases such as obesity. The concern with low-calorie and low-sugar content products has been increasing due to increased interest in health, campaign for reducing saccharides, and the like. As one of solutions to solve adult diseases, obesity, etc. which have become a problem in the world recently, various policies for reducing saccharide consumption of their own nation in many countries including Korea have been implemented. Saccharides include monosaccharides or disaccharides such as glucose (DPI) and maltose (DP2), and specifically, include five kinds of saccharides including sucrose, fructose, glucose, maltose, and lactose.

As the tendency that consumers purchase products while checking the saccharide content in the nutritional composition table of the product is gradually increasing, processed food manufacturers are struggling to reduce the amount of glucose syrup, maltose syrup, etc., having a high saccharide content, in an attempt to reduce the saccharide content. In consumer products, the sum of monosaccharide and disaccharide is indicated as saccharide.

However, these substances are used as extenders, sweeteners, texture improvers and viscosity modifiers in products, and they occupy a very large proportion, so it is difficult to replace them. In particular, polymers such as gums and pectins can be used for viscosity control, but can occur many costs.

On the other hand, there is an urgent need to develop a mixed saccharide that achieves a high viscosity while reducing the saccharide in starch syrup itself. In order to solve these problems, a saccharide syrup having a substantially lower saccharide content, but having a sweet taste and low saccharide content similar to those of conventional products is required.

Allulose syrup has received much interest as a saccharide substitute because it has calories close to approximately zero while having a sweetness similar to that of sugar. However, the allulose syrup has a problem that the viscosity is low compared to starch syrup, which is used as a common sweetener. Therefore, there is a problem that it is not easy to adjust the amount of addition, and when cooking food, it is not in harmony with other ingredients, which makes it less convenient to use and poor cooking compatibility. Therefore, efforts to control the viscosity of allulose syrup in the field of food technology are continuously underway, but these issues remains a challenge that has yet to be resolved.

### DETAILED DESCRIPTION OF THE INVENTION

### Technical Problem

The present disclosure has been devised to solve the above-mentioned problems, and thus, it is an object of the present disclosure to provide a mixed saccharide composition containing oligosaccharides, which is easily controlled to viscosity, have good flavor expression, and give a soft mouthfeel, by using saccharide syrup with a high content of maltotetraose.

The mixed saccharide composition according to the present disclosure may be used for food, food additives, beverage or beverage additives, powder type emulsion compositions, and the like, including conventional glucose syrup, maltose syrup and the like.

The mixed saccharide composition according to the present disclosure comprises a saccharide syrup with a high content of maltotetraose, has low saccharide content and calories than the conventional maltooligosaccharides, glucose syrup, maltose syrup, and low DE glucose syrup, and achieves a viscosity level equivalent to that of conventional starch syrup.

The mixed saccharide composition according to an embodiment of the present disclosure may be powdery form, has a higher glass transition temperature than allulose, so as to be easily powdered, and has a lower glass transition temperature than the conventional maltooligosaccharide powder, thereby achieving a rapid dissolution rate.

### Technical Solution

An embodiment of the present disclosure relates to a mixed saccharide composition comprising a maltooligosaccharide-containing saccharide and allulose. The mixed saccharide composition according to an embodiment of the present disclosure includes maltooligosaccharide and allulose and thus, can achieve an equivalent level of viscosity as a conventional starch syrup, while reducing saccharides compared to a conventional starch syrup.

The maltooligosaccharide-containing saccharide does not contain allulose. The maltooligosaccharide-containing saccharide may include 30 to 60% by weight of maltotetraose (G4) based on 100% by weight of the solid content of the saccharide.

The maltooligosaccharide-containing saccharide may include 30 to 60% by weight of maltotetraose (G4) and 25 to 65% by weight of saccharides having DP8 or higher based on 100% by weight of the solid content of the saccharide.

The maltooligosaccharide-containing saccharide contains 30 to 60% by weight of maltotetraose (G4) and 25 to 55% by weight of saccharides with DP10 or higher, based on 100% by weight of the solid content of the saccharide.

The maltooligosaccharide-containing saccharide contains 30 to 60% by weight of maltotetraose (G4) and 25 to 55% by weight of saccharides with DP10 or higher, based on 100% by weight of the solid content of the saccharide, and the content of the remaining saccharide may be 15 to 45% by weight.

The maltooligosaccharide-containing saccharides contained in the mixed saccharide composition according to an embodiment of the present disclosure may be contained in an amount of 10 to 90% by weight, or 35 to 90% by weight, based on 100% by weight of the solid content of the mixed saccharide composition.

The allulose contained in the mixed saccharide composition according to an embodiment of the present disclosure may be contained in an amount of 10 to 90% by weight, or 10 to 65% by weight based on 100% by weight of the solid content of the mixed saccharide composition.

The mixed saccharide composition according to an embodiment of the present disclosure may have a viscosity of 500 to 4800 cps as measured at a temperature of 25° C.

The mixed saccharide composition according to an embodiment of the present disclosure may not contain a thickener. That is, the mixed saccharide composition according to an embodiment of the present disclosure solves the problem of low viscosity of the conventional allulose, even without including a thickener, and thus, can achieve an equivalent level of viscosity as starch syrup.

The maltooligosaccharide contained in the mixed saccharide composition according to an embodiment of the present disclosure may be contained in the form of a maltooligosaccharide-containing syrup. The maltooligosaccharide-containing syrup may be a maltooligosaccharide-containing syrup having a DE (dextrose equivalent) of 13 to 24. The maltotetraose (G4) content of the maltooligosaccharide-containing syrup may be 30 to 60% by weight based on the solid content. The maltooligosaccharide-containing syrup may have a viscosity of 4,000 to 5,500 cps as measured under the temperature condition of 25° C.

Another embodiment of the present disclosure relates to a method for preparing a mixed saccharide composition comprising a step of preparing a mixed saccharide syrup by mixing a maltooligosaccharide-containing syrup containing 30 to 60% by weight of maltotetraose (G4) based on the solid content of the saccharide, with an allulose syrup to prepare a mixed saccharide syrup. The preparation method may further include the step of powdering the mixed saccharide syrup by spray-drying. The spray-drying may be performed at a temperature lower than the glass transition temperature of the mixed saccharide syrup.

Another embodiment of the present disclosure relates to a food composition comprising a mixed saccharide composition comprising a maltooligosaccharide-containing saccharide and allulose.

Hereinafter, the present disclosure will be described in detail.

The mixed saccharide composition according to an embodiment of the present disclosure may include maltooligosaccharide and allulose.

Herein, unless otherwise stated, the term “saccharide reduction” means that the content of monosaccharides such as glucose, fructose, sucrose, etc. and disaccharides which are known to increase the risk of occurrence of obesity, diabetes, cardiovascular diseases, other various adult diseases when overdosed is lowered, and in this case, the “saccharide” does not include rare saccharides such as allulose and the like.

The “maltooligosaccharide”, which is the active ingredient of the mixed saccharide composition according to the present disclosure, is a functional saccharide widely used in the food field, such as protein denaturation prevention effect, food masking effect, and soft food texture imparting. The maltooligosaccharide is mainly used as food additives, and thereby is effective in increasing the viscosity and moisturizing properties of foods, controlling physical properties such as the freezing point drop or osmotic pressure, as well as controlling sweetness and preventing browning. In a purified state, it is also used as a substrate for analysis of serum amylase.

Typically, the maltooligosaccharide indicates that the sum of maltotriose (G3), maltotetraose (G4), maltopentaose (G5), maltohexaose (G6), maltopentaose (G7) and the like is 40% or more, 50% or more, 60% or more, 70% or more, 80% or more, 90% or more, 95% or more, 99% or more, or 100% by weight relative to 1 g of the syrup.

The maltooligosaccharide according to the present disclosure may preferably be characterized in that maltotetraose (G4) is contained at a high content.

The “maltooligosaccharide” may be included as a maltooligosaccharide-containing saccharide, and the maltooligosaccharide-containing saccharide may mean a maltooligosaccharide-containing saccharide in which the sum of G3 to G7 is 40% by weight or more, 50% by weight or more, 60% by weight or more, 70% by weight or more, 80% by weight or more, 90% by weight or more, 95% by weight or more, or 99% by weight or more, or 100% by weight, based on the solid content of the saccharide, but is not limited thereto. The maltooligosaccharide-containing saccharide may be a maltooligosaccharide-containing syrup. The maltooligosaccharide-containing saccharide includes monosaccharides and disaccharides, G3 to G7 saccharides, and G8 or higher saccharides, and the monosaccharide does not contain allulose.

In an embodiment of the present disclosure, the maltooligosaccharide-containing saccharide may be a maltotetraose-containing saccharide containing maltotetraose in a specific content or more, and for example, it may mean a saccharide containing 30 to 60% by weight of maltotetraose (G4), but is not limited thereto. For example, the lower limit of the maltotetraose (G4) content of the maltooligosaccharide-containing saccharide may be 10% by weight or more, 15% by weight or more, 20% by weight or more, 25% by weight or more, 30% by weight or more, 33% by weight or more, 35% by weight or more, 37% by weight or more, 40% by weight or more, 45% by weight or more, 46% by weight or more, 50% by weight or more, 60% by weight or more, or 70% by weight or more, and the upper limit may be 99% by weight or less, 90% by weight or less, 80% by weight or less, 70% by weight or less, 60% by weight or less, 55% by weight or less, or 50% by weight or less, based on 100% by weight of the solid content of the saccharide. The content of maltotetraose (G4) may be set by a combination of the lower limit and the upper limit. For example, the maltotetraose (G4) solid content of the maltooligosaccharide-containing syrup may be 30 to 99% by weight, 30 to 90% by weight, 30 to 80% by weight, 30 to 70% by weight, 30 to 60% by weight, 40 to 99% by weight, 40 to 90% by weight, 40 to 80% by weight, 40 to 70% by weight, 40 to 60% by weight, 50 to 99% by weight, 50 to 90% by weight, 50 to 80% by weight, 50 to 70% by weight, 50 to 60% by weight, 60 to 99% by weight, 60 to 90% by weight %, 60 to 80% by weight, 60 to 70% by weight, 70 to 99% by weight, 70 to 90% by weight, or 70 to 80% by weight

The lower limit of the content of saccharides with a degree of polymerization (DP) of 8 or higher contained in the maltooligosaccharide-containing saccharide, maltooligosaccharide-containing syrup, or maltotetraose syrup according to an embodiment of the present disclosure may be 15% by weight or more, 20% by weight or more, 25% by weight or more, 27% by weight or more, 30% by weight or more, or 33% by weight or more, and the upper limit may be 65% by weight or less, 60% by weight or less, 55% by weight or less, 53% by weight or less, 50% by weight or less, 47% by weight or less, 45% by weight or less, 40% by weight or less, or 37% by weight or less, based on 100% by weight of the total solid content of the saccharide. The content of the saccharide with DP 8 or higher may be set by a combination of the lower limit and the upper limit. For example, the content of the saccharide with DP 8 or higher in the maltooligosaccharide-containing saccharide may be 25 to 60% by weight, for example, 25% to 60% by weight, 30 to 60% by weight, 25 to 55% by weight, 25 to 50% by weight, 30 to 50% by weight, 25 to 40% by weight.

The lower limit of the content of the saccharide with a degree of polymerization (DP) of 10 or more contained in the maltooligosaccharide-containing saccharide, maltooligosaccharide-containing syrup, or maltotetraose syrup according to an embodiment of the present disclosure may be 15% by weight or more, 20% by weight or more, 25% by weight or more, 27% by weight or more, 30% by weight or more, or 33% by weight or more, and the upper limit may be 55% by weight or less, 53% by weight or less, 50% by weight or less, 47% by weight or less, or 45% by weight or less, based on 100% by weight of the total solid content of the saccharide, and the content of the saccharide with the degree of polymerization (DP) of 10 or more may be set by a combination of the lower limit and the upper limit. For example, the content of the saccharide with DP 10 or higher in the maltooligosaccharide-containing saccharide may be 25 to 55% by weight, for example, 25 to 35% by weight, 35 to 45% by weight, or 45 to 55% by weight.

In a specific embodiment of the present disclosure, the maltooligosaccharide-containing saccharide may be configured such that based on 100% by weight of the solid content contained in the saccharide, the content of maltotetraose is 30 to 60% by weight, the content of the saccharide with DP8 or higher is 15 to 60% by weight, and the content of G3 to G7 is 40 to 85% by weight.

As the content of the saccharide with DP 8 or higher, or saccharides with DP10 or higher is higher in the content of the maltooligosaccharide-containing saccharide or the maltotetraose syrup, it has the effect of increased viscosity and decreased solubility, and also has the effect of increased freezing point during the production of ice cream. That is, the G4 content is higher and as the content of the saccharide with DP 8 or higher or saccharides with DP10 or higher is lower in an oligosaccharide syrup, for example maltotetraose syrup, the freezing point is further decreased and the hygroscopicity is lowered, thereby improving the quality and storage stability.

The maltooligosaccharide, maltooligosaccharide-containing saccharide, maltooligosaccharide-containing syrup, or maltotetraose syrup may have a viscosity of 4,000 to 5,500 cps, 4,000 to 5,200 cps, 4,000 to 5,000 cps, 4,000 to 4,800 cps, 4,000 to 4,500 cps, 4,200 to 5,500 cps, 4,200 to 5,200 cps, 4,200 to 5,000 cps, 4,200 to 4,800 cps, 4,200 to 4,500 cps, 4,500 to 5,500 cps, 4,500 to 5,200 cps, 4,500 to 5,000 cps, 4,500 to 4,800 cps, 4,800 to 5,500 cps, 4,800 to 5,200 cps, 4,800 to 5,000 cps, 5,000 to 5,500 cps, or 5,000 to 5,200 cps as measured under the temperature condition of 25° C.

Maltooligosaccharide is produced by a liquefaction reaction and a saccharification reaction of starch. The maltooligosaccharide according to an embodiment of the present disclosure may be produced by treating a starchy raw material with alpha-amylase as a liquefying enzyme and a maltotetraose-producing amylase as a saccharifying enzyme. Maltotetraose-producing amylase (G4-amylase) is a type of alpha-amylase that decomposes alpha 1,4-glucoside bonds, and generally has a characteristic of decomposing into an exo form. The exo-type alpha-amylase produced by some microorganisms has specificity that produces maltose, maltotriose, maltotetraose, maltopentaose, and maltohexaose. For example, amylase derived from Pseudomonas stutzeri PERM BP-1682 or derived from Pseudomonas saccharophila, or the equivalent level of commercially available enzymes may be used. DE is the dextrose equivalent, which is an index indicating the extent of starch hydrolysis.

The saccharification reaction is a step for decomposing maltodextrin, and glucoamylases and/or maltogenic alpha-amylases are used to hydrolyze the non-reducing end of maltodextrin after the saccharide liquefaction step to produce D-glucose, maltose, and isomaltose. In addition, the maltotetraose-producing amylase (G4-amylase) is an enzyme that hydrolyzes starch to form various maltooligosaccharides, and in particular, is an enzyme that mainly produces maltotetraose composed of tetrasaccharides.

The maltooligosaccharide according to the present disclosure may have a DE (dextrose equivalent) of 13 to 24. More preferably, the maltooligosaccharide according to the present disclosure may have a DE (dextrose equivalent) of 20 to 24.

The mixed saccharide composition according to the present disclosure may have a viscosity equivalent to that of conventional starch syrup. For example, the mixed saccharide composition according to the present disclosure may have a viscosity measured at a temperature of 25° C. of 500 to 5000 cps, 500 to 4800 cps, 500 to 4500 cps, 500 to 4000 cps, 500 to 3,500 cps, 500 to 3,000 cps, 500 to 2,500 cps, 500 to 2,000 cps, 700 to 5000 cps, 700 to 4800 cps, 700 to 4500 cps, 700 to 4000 cps, 700 to 3,500 cps, 700 to 3,000 cps, 700 to 2,500 cps, or 700 to 2,000 cps. For example, the viscosity measured at a temperature of 25° C. may be 1,500 to 2,500 cps. For example, the viscosity measured at a temperature of 25° C. may be 1,500 to 2,000 cps. Therefore, the mixed saccharide composition according to an embodiment of the present disclosure may replace some or all of the starch syrup contained in foods.

The mixed saccharide composition according to the present disclosure may be characterized by having a lower calorie than conventional starch syrup. For example, the mixed saccharide composition according to the present disclosure has a calorie of less than 4 kcal/g, 3.8 kcal/g or less, 3.6 kcal/g or less, 3.5 kcal/g or less, 3.4 kcal/g or less, 3.3 kcal/g or less, 3.2 kcal/g or less, or 3 kcal/g or less. More preferably, the mixed saccharide composition according to the present disclosure may have a calorie of 3.5 kcal/g or less. More preferably, the mixed saccharide composition according to the present disclosure may have a calorie of 3.2 kcal/g or less.

The mixed saccharide composition according to the present disclosure may have a DE (dextrose equivalent) of 20 to 60, 20 to 55, 20 to 50, 20 to 45, 20 to 40, 25 to 60, 25 to 55, 25 to 50, 25 to 45, 25 to 40, 30 to 60, 30 to 55, 30 to 50, 30 to 45, or 30 to 40. The mixed saccharide composition according to an embodiment of the present disclosure has a glass transition temperature (Tg) of 0 to 95° C., 0 to 90° C., 0 to 80° C., 0 to 70° C., 0 to 65° C., 10 to 95° C., 10 to 90° C., 10 to 80° C., 10 to 70° C., 10 to 65° C., 20 to 95° C., 20 to 90° C., 20 to 80° C., 20 to 70° C., 20 to 65° C., 30 to 95° C., 30 to 90° C., 30 to 80° C., 30 to 70° C., 30 to 65° C., 40 to 95° C., 40 to 90° C., 40 to 80° C., 40 to 70° C., 40 to 65° C., 45 to 95° C., 45 to 90° C., 45 to 80° C., 45 to 70° C., or 45 to 65° C. The mixed saccharide composition may be in the form of a powder.

There is a demand for allulose crystals and powders, but it is very difficult to prepare powdered products in a liquid state by a method other than the crystallization method. Allulose can be prepared by performing a chemical method or a biological method, but due to the low content of allulose in the product, purification and concentration steps can be performed to increase the purity of allulose and crystallize the allulose. Further, in the production of allulose, unresolved problems remain in the purification step, purification yield, crystallization yields, and the like.

Generally, as the powder particles of saccharides is finer, the flowability is poorer. Thus, the use convenience which occurs in the processing is reduced. Allulose is highly hygroscopic and thus hardly maintains a powdery state. The powdered sweetener of the present disclosure has the advantage that the dispersibility and flowability are improved, while being less affected by water, thereby being capable of packaging in various types.

The powdering process may be performed by a method such as spray-drying or vacuum drying. However, water evaporates under conditions of temperature and pressure at which water can evaporate, that is, under high temperature and vacuum (or reduced pressure) conditions to form amorphous powder particles. Preferably, the powdered sweetener composition according to the present disclosure may be a spray-dried product prepared by spray-drying a liquid product containing allulose and a powdered auxiliary ingredient. As used herein, the spray-drying is a drying method in which a liquid product is sprayed with hot air and dispersed to rapidly evaporate water while being conveyed to the hot air, and dried to obtain a powder. For example, it may be a single-stage method or a multi-stage method.

Generally, materials used as a liquid product, which is raw material to be used for spray-drying, have an intrinsic glass transition temperature (Tg) of the material itself. However, at a temperature of Tg value or higher, the material changes to a glass transition state, that is, a sticky and elastic softened state. Therefore, when drying is performed at a temperature equal to or higher than Tg value, the powder has a sticky property and thus, is difficult to be recovered as a dry powder. Therefore, especially in the spray-drying process, spray-drying must be performed at a temperature equal to or lower than Tg value. Further, it is important to set the outlet temperature to a temperature equal to or lower than Tg value and to adjust the process parameters so that water can evaporate. Generally, saccharides such as dextrin have higher Tg values as they contain more molecules with high molecular weight. Thus, the powdering is well performed at a high outlet temperature where evaporation of water is easy in the spray-drying process. On the other hand, as the powder has higher saccharide content, it has lower glass transition temperature and particularly has an advantage of being quickly dissolved in liquid substances. This characteristic has the advantage of being easy to use in powder products that are dissolved in a low temperature state and ingested.

However, allulose has a sub-zero glass transition temperature (−5.5° C.), and thus has a very low Tg value compared to other saccharides. In order to prepare allulose powder by the spray-drying process, when the internal temperature or the outlet temperature of the powdering device is set and dried to a temperature below Tg value, water hardly evaporates at a temperature equal to or lower than Tg value, and thus, drying is not properly performed. Therefore, it is very difficult for allulose to form powdery particles by a powdering process as a single component.

Further, maltooligosaccharides have a high glass transition temperature, and thus have a problem that the dissolution rate is slower than that of other saccharides. Therefore, when used as a powder, there is a problem that it is difficult to dissolve in a liquid substance, thereby having a problem that it can be used for very small number of processed foods.

To solve these problems, an embodiment of the present disclosure provides a mixed saccharide composition comprising allulose and maltooligosaccharide, and can provide a mixed saccharide powder with easy powdering and high dissolution rate.

The mixed saccharide composition according to an embodiment of the present disclosure may be in the form of a powder, and the mixed saccharide powder has higher storage stability than liquid syrup and has the effect of extending the shelf-life period, and it has the advantage of being easier to handle than liquid products. In particular, in the case of the mixed saccharide composition in powdery form, it can be powdered by being mixed with components desired by the product purchaser, and thus, the demand of processed food manufacturers for the powder is increasing. Further, in the case of companies that sells products, they can dominate the market competition, since they can used their own products.

The dissolution rate of the mixed saccharide composition according to an embodiment of the present disclosure may be more than 1 to 10 times, more than 1 to 9 times, more than 1 to 8 times, more than 1 to 7 times, more than 1 to 6 times, more than 1 to 5 times, more than 1 to 4 times, more than 1 to 3 times, more than 1 to 2 times, more than 1 to 1.8 times, 1.05 to 10 times, 1.05 to 9 times, 1.05 to 8 times, 1.05 to 7 times, 1.05 to 6 times, 1.05 to 5 times, 1.05 to 4 times, 1.05 to 3 times, 1.05 to 2 times, or 1.05 to 1.8 times, compared to the dissolution rate of the maltooligosaccharide powder containing no allulose (control group).

The dissolution rate of the mixed saccharide composition according to an embodiment of the present disclosure may be more than 100%, 105% or more, 109% or more, 110% or more, 120% or more, 125% or more, 130% or more, 140% or more, 150% or more, 160% or more, or 170% or more of the dissolution rate of the maltooligosaccharide powder containing no allulose (control group). At this time, the upper limit of the dissolution rate may be 1000% or less, 900% or less, 800% or less, 700% or less, 600% or less, 500% or less, 400% or less, 300% or less, 200% or less, 190% or less, or 180% or less of the dissolution rate of the control group.

The dissolution rate may be a rate obtained by measuring the time required for 20 g of the mixed saccharide powder composition or the maltooligosaccharide powder (control group) to be dissolved in 80 g of water, and converting into the inverse number of the measured dissolution time. Therefore, if the dissolution time is short, it means that the dissolution rate is high.

The time required for 20 g of the mixed saccharide composition according to an embodiment of the invention to be dissolved in 80 g of water may be less than 240 seconds, 230 seconds or less, 220 seconds or less, 210 seconds or less, 200 seconds or less, 190 seconds or less, 180 seconds or less, 170 seconds or less, 160 seconds or less, 150 seconds or less, or 140 seconds or less. The time required for dissolution may be the time required for the powder to dissolve in a solvent and reach a normal state.

The lower time limit required for the dissolution may be 50 second or higher, 60 second or higher, 70 second or higher, 80 second or higher, 90 second or higher, 100 second or higher, 110 second or higher, 120 second or higher, 130 second or higher or 140 second or higher.

The time required for 20 g of the mixed saccharide composition according to an embodiment of the invention to be dissolved in 80 g of water may be less than 100%, 99% or lower, 98% or lower, 97% or lower, 96% or lower, 95% or lower, 94% or lower, 93% or lower, 92% or lower, 90% or lower, 80% or lower, 70% or lower, or 60% or lower based on the time required for 20 g of the mixed saccharide composition of control group to be dissolved in 80 g of water. The control group is a maltooligosaccharide powder containing no allulose.

The lower limit of the content of maltooligosaccharide or maltooligosaccharide-containing saccharide in the mixed saccharide composition according to the present disclosure may be 1% by weight or more, 20% by weight or more, 25% by weight or more, 30% by weight or more, 35% by weight or more, 40% by weight or more, 50% by weight or more, 55% by weight or more, 60% by weight or more, 65% by weight or more, 70% by weight or more, 75% by weight or more, 80% by weight or more, or 83% by weight, and the upper limit may be 99.9% by weight or less, 99% by weight or less, 95% by weight or less, 90% by weight or less, 87% by weight or less, based on 100% by weight of the solid content of the mixed saccharide composition. For example, it may have a combined numerical range of the upper limit and the lower limit.

The mixed saccharide composition of the present disclosure contains allulose, and the lower limit of the content of allulose in the mixed saccharide composition may be 0.1% by weight or more, 1% by weight or more, 5% by weight or more, 10% by weight or more, 15% by weight or more, 20% by weight or more, 25% by weight or more, 30% by weight or more, 35% by weight or more, or 40% by weight or more, and the upper limit may be 99% by weight or less, 90% by weight or less, 80% by weight or less, 70% by weight or less, 65% by weight or less, 60% by weight or less, 55% by weight or less, 50% by weight or less, 45% by weight or less, 40% by weight or less, 35% by weight or less, 30% by weight or less, 25% by weight or less, 20% by weight or less, based on 100% by weight of the total solid content of the mixed saccharide or the solid content of the saccharide. The content of allulose may be set by a combination of the lower limit and the upper limit. As an example, the mixed saccharide composition according to the present disclosure may contain 10 to 65% by weight of allulose, based on 100% by weight of solid content. As an example, the mixed saccharide composition according to the present disclosure may contain allulose in an amount of 10 to 45% by weight, 15 to 45% by weight, 20 to 45% by weight, 25 to 45% by weight, 10 to 40% by weight, 15 to 40% by weight, 20 to 40% by weight, 25 to 40% by weight of allulose, 10 to 35% by weight, 15 to 35% by weight, 20 to 35% by weight, or 25 to 35% by weight, based on 100% by weight of solid content.

The mixed saccharide composition according to an embodiment of the present disclosure includes maltooligosaccharide-containing saccharides and allulose, and the maltooligosaccharide-containing saccharide may be included in a larger amount than the allulose. That is, the solid content of the maltooligosaccharide-containing saccharide may be more than 1 times of the solid content of the allulose, but not limited thereto. It will be clear to those skilled in the art that each content can be appropriately adjusted according to the intended purpose.

The allulose may be prepared by various methods, and preferably, it may be prepared by a biological method, for example a microbial enzyme reaction. For example, the allulose may be an allulose-containing mixed saccharides or may be obtained therefrom, and the mixed saccharides may be mixed saccharides prepared by reacting one or more selected from the group consisting of an allulose epimerase, a microbial cell of a strain producing the epimerase, a culture of the strain, a lysate of the strain, and an extract of the lysate or culture with a fructose-containing raw material, or be obtained therefrom.

The mixed saccharide syrup may be mixed saccharides containing 2 to 55 parts by weight of allulose, 30 to 80 parts by weight of fructose, 2 to 60 parts by weight of glucose and 0 to 15 parts by weight of oligosaccharide, based on 100 parts by weight of the solid content of the saccharides, and the allulose syrup may be obtained via separation, purification and concentration processes from the mixed saccharides.

In an embodiment of the present disclosure, the allulose syrup passing through separation and purification processes may be an allulose-containing syrup which has an electrical conductivity of 1 to 50 μS/cm, and is colorless or light-yellow liquid having sweetness and contains containing 5% by weight or more or 10% by weight or more of allulose. For example the allulose-containing syrup may contain allulose in an amount of 5 to 99.9% by weight, 5 to 97% by weight, 5 to 95% by weight, 5 to 93% by weight, 5 to 90% by weight, 5 to 85% by weight, 5 to 80% by weight, 5 to 50% by weight, 5 to 30% by weight, 6.5 to 99.9% by weight, 6.5 to 97% by weight, 6.5 to 95% by weight, 6.5 to 93% by weight, 6.5 to 90% by weight, 6.5 to 85% by weight, 6.5 to 80% by weight, 6.5 to 50% by weight, 6.5 to 30% by weight, 9 to 99.9% by weight, 9 to 97% by weight, 9 to 95% by weight, 9 to 93% by weight, 9 to 90% by weight, 9 to 85% by weight, 9 to 80% by weight, 9 to 50% by weight, 9 to 30% by weight, 9 to 25% by weight, 9 to 20% by weight, 50 to 99.9% by weight, 50 to 97% by weight, 50 to 95% by weight, 50 to 93% by weight, 50 to 90% by weight, 50 to 85% by weight, 50 to 80% by weight, 70 to 99.9% by weight, 70 to 97% by weight, 70 to 95% by weight, 70 to 93% by weight, 70 to 90% by weight, 70 to 85% by weight, 70 to 80% by weight, 80 to 99.9% by weight, 80 to 97% by weight, 80 to 95% by weight, 80 to 93% by weight, 80 to 90% by weight, 80 to 85% by weight, 90 to 99.9% by weight, 90 to 97% by weight, 90 to 95% by weight, 90 to 93% by weight, 93 to 99.9% by weight, 93 to 97% by weight, 93 to 95% by weight, 95 to 99.9% by weight, or 95 to 97% by weight.

Examples of the allulose-containing syrup used for the present disclosure include 5 to 50% by weight of allulose, 1 to 50% by weight of glucose, and 30 to 70% by weight of fructose, based on 100% by weight of the saccharide solid content of the syrup, and may be a syrup produced from fructose by a biological method. In addition, the allulose-containing syrup may not contain or may further contain oligosaccharides.

In the present disclosure, the “high-intensity sweetener” may include one or more selected from the group consisting of a natural high-intensity sweetener and a synthetic high-intensity sweetener, and specifically, may be one or more selected from the group consisting of steviol glycoside, enzymatically modified stevia, aspartame, stevioside aspartame, acesulfame K, sodium cyclamate, sodium saccharin, sucuralose, dulcin, taumatin, neotame and monelline.

The mixed saccharide composition according to the present disclosure may further contain a high-intensity sweetener in an amount of 0.0001 to 5 parts by weight, 0.0005 to 5 parts by weight, 0.001 to 5 parts by weight, 0.005 to 5 parts by weight, 0.01 to 5 parts by weight, 0.0001 to 1.5 parts by weight, 0.0005 to 1.5 parts by weight, 0.001 to 1.5 parts by weight, 0.005 to 1.5 parts by weight, 0.01 to 1.5 parts by weight, 0.0001 to 1.0 parts by weight, 0.0005 to 1.0 parts by weight, 0.001 to 1.0 parts by weight, 0.005 to 1.0 parts by weight, or 0.01 to 1.0 parts by weight, based on 100 parts by weight of the total solid content.

The mixed saccharide composition according to the present disclosure can be used in foods, beverages, livestock feeds, livestock health/nutrition foods, pharmaceutical products, cosmetics, and the like. The mixed saccharide composition according to the present disclosure has the effects of softening the texture of foods and feeds, increasing the volume, increasing the concentration, preventing crystallization of sugar, and enhancing flavor and sweetness. The mixed saccharide composition according to the present disclosure may replace all or part of the components of ionic starch syrup, malt starch syrup, or low DE glucose syrup used in food.

The mixed saccharide composition according to the present disclosure may not contain a thickener. In the case of allulose syrup having a low viscosity, it is mixed with the maltooligosaccharide according to the present disclosure and thereby, can provide an appropriate range of viscosities that can increase consumer satisfaction without the use of additional thickeners.

The allulose syrup having a low viscosity is being ignored by consumers due to its low viscosity. Even if a thickener is added to increase the viscosity, there is a limit in that it is difficult to achieve sufficient viscosity due to the low solubility of the thickener. In addition, in order to increase the solubility of the thickener, it must be reacted for a long time at high temperature, and in this process, there is a problem that saccharides and other active ingredients are denatured or destroyed. The mixed saccharide composition according to the present disclosure, including allulose syrup and maltooligosaccharide can achieve high viscosity without the addition of a thickener.

Another embodiment of the present disclosure provides a food composition comprising the mixed saccharide composition. The food may be one or more selected from the group consisting of ice cream, coffee mix, dairy products (fermented milk, almond milk), creamer, effervescent vitamin, powdered drinks, soy milk, tea drinks, hard candies, jelly/gummi, cookie, fried cookie, pie and biscuit.

Another embodiment of the present disclosure relates to a method for preparing the mixed saccharide syrup comprising a step of mixing maltooligosaccharide syrup and allulose syrup to prepare a mixed saccharide syrup. The maltooligosaccharide syrup may include 30 to 60% by weight of maltotetraose (G4) based on the solid content of the saccharide.

The method for preparing the mixed saccharide composition may further include a step of spray-drying and powdering the mixed saccharide syrup after the step of preparing the mixed saccharide syrup.

In the spray-drying, a liquid product to be sprayed may be sprayed by various spraying means, such as a disk or a nozzle, and dried by blowing hot air inside a dryer. Examples of atomizers of the spray dryer include a two-fluid nozzle, a pressure nozzle, and a rotary atomizer.

### Advantageous Effects

The mixed saccharide composition according to the present disclosure not only has low calories by replacing the saccharide contained in the conventional starch syrup, but also has the effect of maintaining high viscosity and thus achieving both calorie reduction and high viscosity.

### BRIEF DESCRIPTION OF THE DRAWINGS

FIG. 1 is a graph showing the dissolution time of the mixed saccharide powder according to an embodiment of the present disclosure.

FIG. 2 is a view showing the results of the sensory evaluation of an ice cream to which the mixed saccharides according to an embodiment of the present disclosure is applied.

### DETAILED DESCRIPTION OF THE EMBODIMENTS

Hereinafter, the present disclosure will be described in more detail with reference to the following examples, but the following examples are provided as only preferable examples of the present disclosure only and are not intended to limit the scope of the present disclosure.

### Preparation Example 1: Preparation of Maltooligosaccharide

7000 g of corn starch was mixed with 13000 g of water, and then the mixture was subjected to high temperature liquefaction reaction at 110° C. through a hydroheater, and then passed through a hydroheater at 130° C. to 140° C. to deactivate the liquefying enzyme. After that, the temperature was lowered to 61° C. through a heat exchanger, and then high content maltotetraose was hydrolyzed using a heat-resistant a-amylase derived from Pseudomonas stutzeri. and heated to 80° C. at DE 20 to 22 when the reaction was completed. An activated carbon was added in an amount of 0.1 to 0.8% by weight based on the solid content, and the mixture was stirred for 30 minutes or more. Thereafter, the activated carbon was removed through a filter press, followed by ion purification and concentration to obtain 9600 g of maltotetraose syrup. The saccharide composition of the obtained maltooligosaccharide is shown as solid content % by weight in Table 1. “monosaccharide and disaccharide” listed in Table 1 represent the contents of all monosaccharides and disaccharides contained in maltooligosaccharides.

TABLE 1

| | | Saccharide composition (solid content % by weight) | | | | | | |
|---|---|---|---|---|---|---|---|---|
| | | octa- | malto-oligosaccharide | | | | | mono- |
| | | saccharide | Hepta- | hexa- | penta- | tetra- | tri- | saccharide and |
| Item | DE | or higher | saccharide | saccharide | saccharide | saccharide | saccharide | disaccharide |
| Preparation | 21 | 35 | 0 | 0.3 | 2.6 | 46.8 | 8.6 | 6.7 |
| Example 1 | | | | | | | | |
| Comparative | 42.2 | 21.9 | 2 | 3.6 | 5.6 | 6.1 | 17.6 | 43.2 |
| Example 1 | | | | | | | | |
| (Glucose syrup) | | | | | | | | |
| Comparative | 22.5 | 29.1 | 3.3 | 20.1 | 14 | 6.2 | 13.4 | 13.9 |
| Example 2 | | | | | | | | |
| (Low DE | | | | | | | | |
| glucose syrup) | | | | | | | | |

### Comparative Example 1: Glucose Syrup

Glucose syrup (Samyang Corporation, DE 42) was used as a comparative example. Specifically, 1000 g of corn starch was mixed with 2500 g of water, and then the mixture was subjected to a high temperature liquefaction reaction at 110° C. through a hydroheater, and then passed through a hydroheater at 130° C. to 140° C. to deactivate the liquefying enzyme. After that, the temperature was lowered to 61° C. through a heat exchanger, and then the resulting mixture was allowed to react up to DE 40 to 45 using Maltogenase (Novozymes) and Pullulanase (Novozymes, Promozyme D2) as the saccharifying enzymes. The activated carbon was added in an amount of 0.1 to 0.8 wt % based on the solid content and was stirred for 30 minutes or more. Then, the activated carbon was removed through a filter press, followed by ion purification and concentration to obtain 2000 g of Glucose syrup. The saccharide composition of the obtained glucose syrup is shown as solid content wt % in Table 2 below.

### Comparative Example 2: Low DE Glucose Syrup

Low DE glucose syrup (Samyang Corporation, DE 24) was used as a comparative example. Specifically, 1000 g of corn starch was mixed with 2500 g of water, and then the mixture was subjected to a high temperature liquefaction reaction at 110° C. through a hydroheater, and then passed through a hydroheater at 130° C. to 140° C. to deactivate the liquefying enzyme. After that, the temperature was lowered to 61° C. through a heat exchanger, and then the resulting mixture was allowed to react up to DE 20 to 24 using alpha-amylase (Novozyme, Liquozyme Supra) as a liquefying enzyme used in the liquefaction reaction. The activated carbon was added in an amount of 0.1 to 0.8 wt % relative to the solid content and was stirred for 30 minutes or more. Then, the activated carbon was removed through a filter press, followed by ion purification and concentration to obtain 2000 g of low DE glucose syrup. The saccharide composition of the obtained Low DE glucose syrup is shown as solid content wt % in Table 2.

### Examples 1 to 8: Preparation of Liquid Mixed Saccharides

The maltooligosaccharide prepared in Preparation Example 1 was fractionated into six by 1000 g each, and then was mixed with 70 Brix allulose syrup containing 96% by weight of allulose. Based on the solid content of the total mixed saccharides, the solid content of allulose increased as it went from Example 1 to Example 6, and the final solid content of allulose in the mixed saccharides was mixed to be numerical value as shown in Table 2.

TABLE 2

| | | | Maltooligosaccharid- |
|---|---|---|---|
| | | Allulose content | containing |
| | | of the mixed | saccharides content |
| | Mixed | saccharide | of the mixed |
| | saccharides | (solid content | saccharide (solid |
| Item | DE | % by weight) | content % by weight) |
| Example 1 | 30.1 | 15.4 | 84.6 |
| Example 2 | 32.0 | 18.0 | 82.0 |
| Example 3 | 33.2 | 20.3 | 79.7 |
| Example 4 | 35.1 | 23.0 | 77.0 |
| Example 5 | 36.7 | 25.1 | 74.9 |
| Example 6 | 40.5 | 30.1 | 69.9 |
| Example 7 | 43.3 | 35.1 | 64.9 |
| Example 8 | 46.5 | 40.2 | 59.8 |

### Test Example 1: Viscosity Measurement

The samples of Preparation Example 1, Examples 1 to 8, and Comparative Examples 1 to 2 were stored in a constant temperature water bath at 25° C. for 1 hour, and then the viscosity was measured using Brookfield. Table 3 shows the viscosity of each sample at 25° C.

TABLE 3

| Item | DE | Brix | cps |
|---|---|---|---|
| Example 1 | 30.1 | 71 | 2450 |
| Example 2 | 31.8 | 71 | 2210 |
| Example 3 | 33.2 | 71 | 1932 |
| Example 4 | 35.5 | 71 | 1842 |
| Example 5 | 36.7 | 71 | 1620 |
| Example 6 | 40.5 | 71 | 1320 |
| Example 7 | 43.3 | 71 | 1010 |
| Example 8 | 46.5 | 71 | 715 |
| Comparative Example 1 | 42.6 | 77 | 1550 |
| (Glucose syrup) | | | |
| Comparative Example 2 | 22.4 | 71 | 2000 |
| (Low DE glucose syrup) | | | |
| Preparation Example 1 | 21.1 | 71 | 4900 |
| (maltooligosacchride) | | | |

As a result, it was confirmed that the viscosity range of the mixed saccharides of Examples 1 to 8 encompass the viscosity range of Comparative Examples 1 and 2, so that the mixed saccharides of Examples 1 to 8 can replace a commercially available starch syrup. Since the viscosity changes depending on the mixing ratio of allulose and maltooligosaccharide, the mixing ratio of allulose and maltooligosaccharide can be adjusted to prepare a product having an appropriate viscosity. In particular, even if allulose with low viscosity is used to replace saccharide, the same level of viscosity as that of a conventional starch syrup can be achieved by mixing with allulose, since the viscosity of the maltooligosaccharide of Preparation Example 1 is remarkably high.

### Test Example 2: Measurement of Calorie

Calories were calculated based on the solid content, and the calorie of allulose was calculated as 0.0 kcal/g. The measured calories are shown in Table 4 below.

TABLE 4

| Item | Calorie (kcal/g) |
|---|---|
| Example 1 | 3.40 |
| Example 2 | 3.30 |
| Example 3 | 3.20 |
| Example 4 | 3.10 |
| Example 5 | 3.00 |
| Example 6 | 2.80 |
| Example 7 | 2.60 |
| Example 8 | 2.40 |
| Comparative Example 1 | 4.00 |
| Comparative Example 2 | 4.00 |
| Preparation Example 1 | 4.00 |

As a result, it was confirmed that the mixed saccharide compositions of Examples 1 to 8 had significantly lower calories than Comparative Example 1 and Comparative Example 2, and thus had the effect of reducing calories even while replacing the conventional starch syrup.

### Examples 9 to 13: Preparation of Mixed Saccharide Powder

The mixed saccharide syrups of Examples 1 to 5 was sprayed using a spray dryer (manufactured by GEA Niro, model name: HKC-100-DJ) with two-fluid nozzle type atomizer. The powder was prepared under the condition that the inlet temperature of the hot air was maintained at 160 to 180° C., and the hot air temperature in the spray dryer and the outlet was maintained at 85 to 100° C. Each powdered sample was designated as Examples 9 to 13, respectively.

As a result, it was confirmed that the mixed saccharides containing 25% by weight of allulose was well powdered, and thus, the mixed saccharides according to the present disclosure could be used in either a liquid syrup form or a powder form.

### Test Example 3: Measurement of Glass Transition Temperature (Tg) of Mixed Saccharide Powder

The Tg value of the mixed saccharide powders prepared in Examples 9 to 13 was analyzed using a differential scanning calorimeter. Analysis conditions were set to start at −50° C. and rise to 150° C. by 5° C. per minute. 3 mg of the sample was placed in a pan for DSC sample, sealed, and then analyzed. The maltooligosaccharide mixed saccharides of Preparation Example 1 to which allulose was not added was used as a control group.

As a result, it was confirmed that the Tg value decreased when allulose was mixed. At this time, it was confirmed that when allulose was contained in an amount of 18% to 25% by weight, the Tg value was lowered by about 40 to 60% compared to Preparation Example 1. Therefore, as maltooligosaccharide was mixed with allulose to prepare a mixed saccharide powder, the dissolution rate increases. Therefore, it is expected that the mixed saccharide powder according to an embodiment of the present disclosure can be applied to processed foods that needs to be quickly dissolved in water, such as coffee mix, powdered beverage, and effervescent vitamin

TABLE 5

| Item | Tg (° C.) |
|---|---|
| Example 9 | 61.4 |
| Example 10 | 57.1 |
| Example 11 | 52.4 |
| Example 12 | 49.6 |
| Example 13 | 46.3 |
| Preparation Example 1 | 97.5 |

### Test Example 4: Measurement of Dissolution Rate of Mixed Saccharide Powder

In order to confirm the solubility of the mixed saccharide powder prepared by spray-drying method, the dissolution rate of the maltooligosaccharide and allulose mixed powder sweeteners of Examples 9 to 13 were compared with that of the maltooligosaccharide powder of Preparation Example 1.

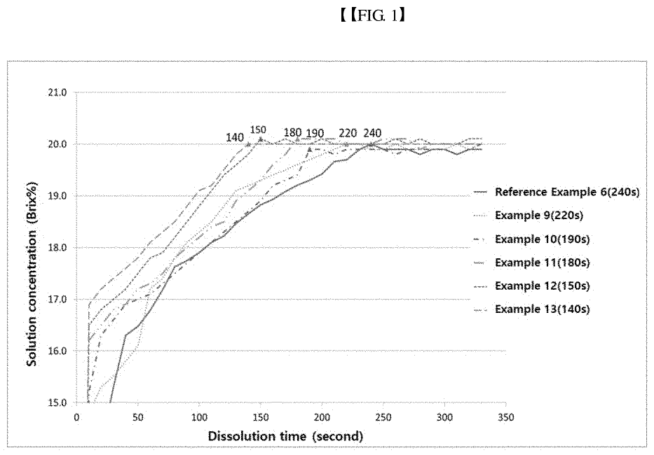

Specifically, using the same beaker and magnetic bar, 20 g of the powder was completely dissolved in 80 g of water at the same stirring speed at room temperature, and the time required for 20 g of the powder be completely dissolved (i.e., the dissolution rate) was measured. The results are shown in Table 6 and FIG. 1. As a result, it was confirmed that the dissolution rate of the mixed saccharides of the present disclosure is fast. Specifically, all the samples showed excellent dissolution rates compared to Preparation Example 1, and in particular, the samples of Examples 13 showed the fastest dissolution rate. In Table 1, the relative dissolution time compared to Preparation Example 1 is a relative value calculated by dividing the dissolution time of each sample by the dissolution time of Preparation Example 1, and a smaller value means a faster dissolution rate compared to Preparation Example 1.

TABLE 6

| | Dissolution | Dissolution time |
|---|---|---|
| | time | relative to that of |
| Item | (second) | Preparation Example 1 |
| Example 9 | 220 | 91.7% |
| Example 10 | 190 | 86.4% |
| Example 11 | 180 | 75.0% |
| Example 12 | 150 | 62.5% |
| Example 13 | 140 | 58.3% |
| Preparation Example 1 | 240 | 100.0% |

### Example 14: Preparation of Ice Cream

The maltotetraose syrup and liquid allulose syrup (allulose content of 96 wt %, 75 brix) of Preparation Example 1 were mixed at a mixing ratio as shown in Table 7 below to prepare a liquid composition for producing ice cream.

Specifically, while raising the temperature of the container in a constant temperature water bath at 40° C., purified water, allulose syrup, maltotetraose syrup, white sugar, frozen milk cream, skim milk powder, cocoa powder, emulsifier and emulsion stabilizer (Cremodan Sim) were added, and the mixture was stirred at 200 rpm for 15 minutes at 65° C., and homogenized with a homogenizer at 5000 rpm for 5 minutes for complete emulsification. The homogenized composition was sterilized at 85° C. for 10 minutes and then cooled with cold water for about 1 hour. The cooled mixture was aged for about 12 to 16 hours. Therefore, an aged liquid composition for the preparation of ice cream was prepared.

The agenda composition was introduced into a quick freezer set at a temperature of −35° C. to −40° C., and the freezer was operated based on the time point when the ice cream temperature was −4° C. The dispersed composition was overrun using a continuous freezer (Tetra: Hoyer Frigus KF 80F1) to prepare an ice cream.

### Comparative Example 3: Preparation of Ice Cream Containing Only Sugar

An emulsified composition containing sugar was prepared by substantially the method and composition as in Example 14, except that in Example 14, only sugar was used without using allulose and maltotetraose syrup.

### Comparative Example 4: Preparation of Ice Cream Containing Allulose Alone

An emulsified composition containing allulose was prepared by substantially the method and composition as in Example 14, except that in Example 14, allulose syrup (allulose content 96 wt %, 76Brix) and sugar (Samyang Corporation, white sugar) were used without using maltotetraose syrup.

The components and contents of the composition for the preparation of ice cream of Example 14 and Comparative Examples 3 to 4 are shown in Table 7 below. The units in Table 7 below are weight %, and allulose and maltotetraose were the weight of the liquid syrup, and maltotetraose syrup was 72 brix.

TABLE 7

| | | Comparative | Comparative |
|---|---|---|---|
| Component | Example 14 | Example 3 | Example 4 |
| Frozen milk cream | 25.0 | 25.0 | 25.0 |
| (MF44%) | | | |
| Skim milk powder | 7.99 | 7.99 | 7.99 |
| White sugar | 14.00 | 16.50 | 14.00 |
| Allulose syrup | 3.10 | — | 3.10 |
| Matotetraose syrup | 0.60 | — | — |
| Emulsion stabilizer | 5.67 | 5.67 | 5.67 |
| Coca powder | 0.41 | 0.41 | 0.41 |
| Distilled water | 43.23 | 44.43 | 44.83 |
| Sum | 100 | 100 | 100 |

### Test Example 5: Sensory Evaluation of Ice Cream

The sensory evaluation (softness of food texture, preference of food texture, off-taste, and off-flavor) was performed on 50 ordinary people by a method of calculating the average value by evaluating each item by a 4-point method.

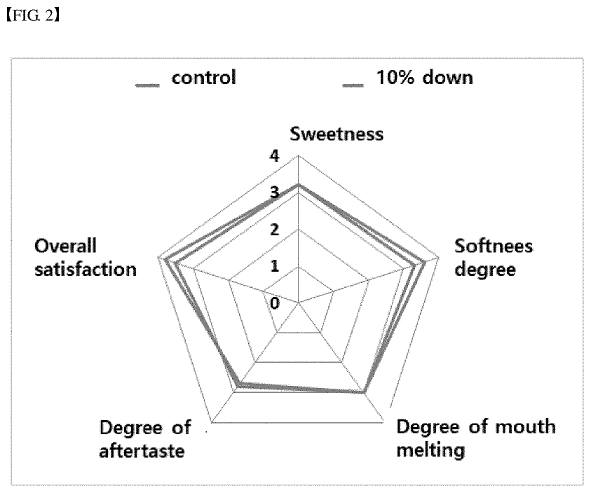

The evaluation values of the sweetness, food texture softness, overall satisfaction of food texture, aftertaste intensity, and degree of melting in mouth (mouth melting) were evaluated as 4 points of perfect score, and the evaluation results are shown in FIG. 2

As a result of sensory evaluation, the ice cream containing maltotetraose syrup and allulose syrup of Example 14 was evaluated more positively than the ice cream containing only sugar (Comparative Example 3) and the ice cream containing allulose and sugar (Comparative Example 4). Especially for the soft texture, the content of maltotetraose syrup with DP10 or more is high, so the mouthfeel is enhanced. By increasing the mouthfeel, it is effective in giving softness in the mouth, and the result of the sensory test also confirmed the soft mouth feel effect of the ice cream applied with allulose. The overall satisfaction of the texture of food was slightly higher in the ice cream of Example 14, but it showed a relatively equivalent degree.

### Test Example 6: Evaluation of Physical Properties of Ice Cream

(1) Evaluation of Overrun

Usually, when making ice cream, as air in the tissue is mixed and dispersed while freezing the composition for making ice cream which is the raw material mixture, thereby properly expanding the raw material mixture, the rate of increase in the increased capacity is referred to as an overrun. Therefore, in the production of ordinary ice cream, a dedicated freezer or maker for ice cream production is used in which a large amount of air is mixed and frozen. The overrun measurement method is calculated according to the following Equation 1 by measuring the weight at a constant volume.

$$Overrun ( \% ) = \frac{Weight of ice cream mix - Weight of ice cream}{Weight of ice cream} \times 100 \qquad [ Equation 1 ]$$

(2) Evaluation of Freezing Point Drop

It was measured using a cryoscope.

It can be said that the overrun values of the ice creams according to Comparative Examples 3 and 4 and Example 14 can be maintained at similar levels as compared with the ice creams using only sugar or using allulose and sugar together.

In relation to the freezing point, it was confirmed that the ice cream of Example 14 using maltotetraose syrup has a significant difference as compared with the ice cream of Comparative Example 3 using sugar. In addition, in the case of the ice cream of Comparative Example 4 using allulose and sugar, the freezing point was lowered, whereas the ice cream of Example 14 containing both allulose and maltotetraose syrup slightly increased the lowered freezing point. Thereby, the present disclosure is a composition for the preparation of ice cream and an ice cream product that complements the freezing point reduction and increased melting rate of ice cream resulting from the inclusion of allulose. Specifically, the present disclosure can improve the low freezing point and melting rate of an emulsion composition containing allulose by using a syrup with a high content of maltotetraose, and can provide an emulsion composition that has low calorie and excellent solubility, and at the same time, have an improved freezing point and melting rate, and a method for preparing the same.

### Example 15: Preparation of Creamer

350 g of hardened coconut oil, 6 g of a mixture of monoglyceride and diglyceride as an emulsifier, and 2 g of sodium stearoyl lactylate were mixed, heated at 65° C. or higher, and mixed to prepare an oil phase. 592 g of each mixed saccharide syrup of Examples 1 to 5 was mixed with 20 g of casein sodium, 21 g of dibasic potassium phosphate, 4 g of potassium polyphosphate and 5 g of sodium silicon aluminate, and stirred at 70° C. to 75° C. or higher to prepare an aqueous phase. The oil phase and the aqueous phase were mixed with a homomixer at 4000 rpm or more for 10 minutes or more. Thereafter, secondary mixing was performed using a homogenizer, and then powder was prepared using spray-drying.

As a result of comparing the hygroscopicity of the prepared powder with that of the glucose syrup of Comparative Example 1 and the low DE glucose syrup of Comparative Example 2, the coffee creamer using the mixed polysaccharide according to one example of the present disclosure had an advantage that it exhibited lower hygroscopicity and could improve the storage stability of the product as compared with the products of Comparative Example.

### Example 16: Preparation of Carbonated Drink

(1) Preparation of Carbonated Drinks Using Mixed Saccharide Powder

The mixed saccharide powder of any one of Examples 9 to 13, sucralose, acesulfame potassium, rebaudioside A, citric acid, lemon lime flavor and purified water were mixed in a mixing ratio of Table 8 below to prepare a drink syrup liquid. The drink syrup liquid was transferred to a tank, and the volume was adjusted with purified water, and then carbon dioxide was injected at a temperature of 4 to 8° C. so that the CO₂ volume (the number of gram equivalents of CO₂ gas dissolved in 22.5 L of carbonated drink) becomes 3.5 to 4.0 volume.

TABLE 8

| | Example | Example | Example | Example | Example |
|---|---|---|---|---|---|
| Component | 16-1 | 16-2 | 16-3 | 16-4 | 16-5 |
| Mixed saccharide powder | 1.000 | 3.00 | 5.00 | 7.00 | 10.00 |
| Sucralose | 0.0085 | 0.0078 | 0.007 | 0.006 | 0.005 |
| Acesulfame potassium | 0.0182 | 0.017 | 0.016 | 0.015 | 0.012 |
| Carbon dioxide | CO₂ volume 3.5~4.0 | | | | |
| Citric acid | 0.120 | 0.120 | 0.120 | 0.120 | 0.120 |
| Lemon lime flavor | 0.085 | 0.085 | 0.085 | 0.085 | 0.085 |
| Distilled | Remaining | Remaining | Remaining | Remaining | Remaining |
| water | amount | amount | amount | amount | amount |
| Sum | 100.000 | 100.000 | 100.000 | 100.000 | 100.00 |

(2) Preparation of Carbonated Drinks Using Mixed Saccharide Syrup

The mixed saccharide syrup of any one of Examples 1 to 5, rebaudioside A, citric acid, lemon lime flavor and purified water were mixed in a mixing ratio of Table 9 below to prepare a drink syrup liquid. The drink syrup liquid was transferred to a tank, and the volume was adjusted with purified water, and then carbon dioxide was injected at a temperature of 4 to 8° C. so that the CO₂ volume (the number of gram equivalents of CO₂ gas dissolved in 22.5 L of carbonated drink) becomes 3.5 to 4.0 volume.

TABLE 9

| | Example | Example | Example | Example | Example |
|---|---|---|---|---|---|
| Component | 16-6 | 16-7 | 16-8 | 16-9 | 16-10 |
| Mixed saccharide | 1.000 | 3.00 | 5.00 | 7.00 | 10.00 |
| syrup | | | | | |
| Rebaudioside A | 0.0400 | 0.0360 | 0.0330 | 0.0290 | 0.0240 |
| Carbon dioxide | CO₂ volume 3.5~4.0 | | | | |
| Citric acid | 0.120 | 0.120 | 0.120 | 0.120 | 0.120 |
| Lemon lime flavor | 0.085 | 0.085 | 0.085 | 0.085 | 0.085 |
| Distilled water | Remaining | Remaining | Remaining | Remaining | Remaining |
| | amount | amount | amount | amount | amount |
| Sum | 100.000 | 100.000 | 100.000 | 100.000 | 100.000 |

### Example 17: Preparation of Energy Drink

An energy drink containing the mixed saccharide syrup or powder prepared in Examples 1 to 13 was prepared.

Specifically, the mixed saccharide syrup or powder was mixed with each ingredients based on the composition and mixing ratio (w/w %) of Table 10 below, and the mixture was stirred, filtered with 120 mesh, and then sterilized for 30 seconds at a temperature of 98° C. This was filtered again through 80 mesh and then filled at 88° C. Then, it was post-sterilized and cooled at a temperature of 85° C. for 15 minutes to prepare an energy drink.

TABLE 10

| | Comparative | Example | Example | Example | Example | Example |
|---|---|---|---|---|---|---|
| Component | Example | 17-1 | 17-2 | 17-3 | 17-4 | 17-5 |
| Mixed saccharide | — | 8.200 | 8.200 | 8.200 | 8.200 | 8.200 |
| Maltitol syrup | 8.200 | — | — | — | — | — |
| Enzymatically | — | — | 0.005 | 0.010 | 0.015 | 0.020 |
| Modified Rutin | | | | | | |
| Vitamin premix | 0.0517 | 0.0517 | 0.0517 | 0.0517 | 0.0517 | 0.0517 |
| Taurine and | 0.832 | 0.832 | 0.832 | 0.832 | 0.832 | 0.832 |
| natural caffein | | | | | | |
| Apple juice | 0.020 | 0.020 | 0.020 | 0.020 | 0.020 | 0.020 |
| concentrate (72bx) | | | | | | |
| Citric anhydride | 0.490 | 0.490 | 0.490 | 0.490 | 0.490 | 0.490 |
| Sucralose | 0.016 | 0.016 | 0.016 | 0.016 | 0.016 | 0.016 |
| Drink flavor | 0.125 | 0.125 | 0.125 | 0.125 | 0.125 | 0.125 |
| Distilled | Remaining | Remaining | Remaining | Remaining | Remaining | Remaining |
| water | amount | amount | amount | amount | amount | amount |
| Sum | 100.000 | 100.000 | 100.000 | 100.000 | 100.000 | 100.000 |

As the vitamin premix, a composition containing 0.0025% by weight of vitamin B6 hydrochloride, 0.0026% by weight of vitamin B2 phosphate ester sodium, 0.0025% by weight of vitamin B1 nitrate, 0.0081% by weight of nicotinic acid amide, and 0.0441% by weight of other ingredients based on 100% by weight of the total composition, was used.

### Example 18: Preparation of Fruit Vegetable Drink

Fruit vegetable drinks containing the mixed saccharide syrup or powder prepared in Examples 1 to 13 were prepared based on the composition (w/w %) shown in Table 11 below.

TABLE 11

| | Example | Example | Example | Example |
|---|---|---|---|---|
| Component | 18-1 | 18-2 | 18-3 | 18-4 |
| Orange juice | 13.850 | 13.850 | 13.850 | 13.850 |
| concentrate(USA, | | | | |
| standard saccharide | | | | |
| content 9) | | | | |
| White sugar | 3.150 | 2.100 | 1.050 | — |
| (Samyang | | | | |
| corporation) | | | | |
| Mixed saccharides | 1.500 | 3.000 | 4.500 | 6.000 |
| of Example 1 to 13 | | | | |
| Isomaltosaccharide | 0.850 | 1.000 | 1.500 | 2.000 |
| (Samyang | | | | |
| corporation) | | | | |
| Citric acid | 0.180 | 0.180 | 0.180 | 0.180 |
| (Samyang | | | | |
| corporation) | | | | |
| Vitamin C | 0.020 | 0.020 | 0.020 | 0.020 |
| (Samyang | | | | |
| corporation) | | | | |
| Rebaudioside A | 0.002 | 0.004 | 0.006 | 0.008 |
| Orange flavor | 0.059 | 0.059 | 0.059 | 0.059 |
| Distilled water | Remaining | Remaining | Remaining | Remaining |
| | amount | amount | amount | amount |
| Sum | 100.00 | 100.00 | 100.00 | 100.00 |

As the isomaltooligosaccharide, isomaltooligosaccharide mixed saccharides having the composition shown in Table 12 (based on 100% by weight of the mixed saccharides solid content) was used.

TABLE 12

| | | | | DP4 to | DP10 or |
|---|---|---|---|---|---|
| Item | DP1 | DP2 | DP3 | DP9 | higher |
| % by weight | 8.1 | 15 | 20 | 32.6 | 24.3 |

1. A mixed saccharide composition comprising a maltooligosaccharide-containing saccharide and allulose, wherein the maltooligosaccharide-containing saccharide contains 30 to 60% by weight of maltotetraose (G4) based on 100% by weight of the solid content of the maltooligosaccharide-containing saccharide.

2. The composition according to claim 1, wherein the maltooligosaccharide-containing saccharide contains 30 to 60% by weight of maltotetraose (G4) and 25 to 65% by weight of saccharides with DP8 or higher based on 100% by weight of the solid content of the maltooligosaccharide-containing saccharide.

3. The composition according to claim 1, wherein the maltooligosaccharide-containing saccharide contains 30 to 60% by weight of maltotetraose (G4) and 25 to 55% by weight of saccharides with DP10 or higher, based on 100% by weight of the solid content of the maltooligosaccharide-containing saccharide.

4. The composition according to claim 1, wherein the maltooligosaccharide-containing saccharide contains 30 to 60% by weight of maltotetraose (G4) and 25 to 55% by weight of saccharide with DP10 or higher, based on 100% by weight of the solid content of the maltooligosaccharide-containing saccharide, and the content of the remaining saccharide is 15 to 45% by weight.

5. The composition according to claim 1, wherein the maltooligosaccharide-containing saccharide is contained in an amount of 10 to 90% by weight based on 100% by weight of the solid content of the mixed saccharide composition.

6. The composition according to claim 1, wherein the allulose is contained in an amount of 10 to 90% by weight based on 100% by weight of the solid content of the mixed saccharide composition.

7. The composition according to claim 1, wherein the mixed saccharide composition has a viscosity of 500 to 4800 cps as measured at a temperature of 25° C.

8. The composition according to claim 1, wherein the composition does not contain a thickener.

9. The composition according to claim 1, wherein the mixed saccharide composition has a calorie of less than 4 kcal/g.

10. The composition according to claim 1, wherein the maltooligosaccharide is contained in the form of a maltooligosaccharide-containing syrup.

11. The composition according to claim 1, wherein the maltooligosaccharide is contained in the form of a maltooligosaccharide-containing syrup having a DE (dextrose equivalent) of 13 to 24.

12. The composition according to claim 10, wherein the maltooligosaccharide-containing syrup has a viscosity of 4,000 to 5,500 cps as measured under the temperature condition of 25° C.

13. The composition according to claim 10, wherein the allulose is provided as an allulose-containing syrup containing allulose in an amount of 5 to 99.9% by weight,

14. The composition according to claim 1, wherein the mixed saccharide composition further contains a high-intensity sweetener in an amount of 0.0001 to 5 parts by weight based on 100 parts by weight of the solid content.

15. (canceled)

16. (canceled)

17. The composition according to claim 1, wherein the composition is in a powder form, and a dissolution rate of the composition is more than 1 to 10 times or less that of the maltooligosaccharide powder.

18. The composition according to claim 1, wherein the composition has a glass transition temperature (Tg) of 0 to 95° C.

19. A food composition comprising the mixed saccharide composition of claim 1.

20. (canceled)

21. A method for preparing a mixed saccharide composition comprising a step of mixing a maltooligosaccharide-containing syrup containing 30 to 60% by weight of maltotetraose (G4) based on the solid content of the saccharide, with an allulose syrup to prepare a mixed saccharide syrup.

22. The method according to claim 21, further comprising a step of spray-drying and powdering the mixed saccharide syrup.

23. The method according to claim 22, wherein the spray-drying is performed at a temperature lower than the glass transition temperature of the mixed saccharide syrup.
